(19) United States
(12) Patent Application Publication
(10) Pub. No.: US 2023/0020486 A1
(43) Pub. Date: Jan. 19, 2023

(54) Stem Cell Delivery

(71) Applicant: University of Miami, Miami, FL (US)

(72) Inventors: Sylvia Daunert, Miami, FL (US); Sapna K. Deo, Miaimi, FL (US); Omaida Velazquez, Miami, FL (US); Zhao-Jun Liu, Miami, FL (US); Prasoon Poozhikun-Nath Mohan, Miami, FL (US); Doyoung Chan, Miami, FL (US); Emre Dikici, Miami, FL (US)

(21) Appl. No.: 17/786,413

(22) PCT Filed: Dec. 21, 2020
(86) PCT No.: PCT/US2020/066413

§ 371 Date: Jun. 16, 2022

Related U.S. Application Data
(60) Provisional application No. 62/950,585, filed on Dec. 19, 2019.

(51) Int. Cl. A61K 9/51; A61K 35/28; A61P 1/16

(52) U.S. Cl. CPC A61K 9/5146; A61K 35/28; A61P 1/16

(57) ABSTRACT

This disclosure relates to systems, compounds and methods for stem cell delivery. More specifically, the disclosure relates a system for promoting tissue regeneration, the system comprising a plurality of stem cells coated with at least one or a plurality of dendrimer nanocarriers that specifically bind to an adhesion molecule. Additionally, the disclosure relates to methods for delivering stem cells to damaged or diseased tissue for stem cell regeneration of the tissue.

### CROSS-REFERENCE TO RELATED APPLICATIONS

This application claims the benefit of U.S. Provisional Patent Application Ser. No. 62/950,585 filed on Dec. 19, 2019, the disclosure of which is explicitly incorporated by reference herein.

### FIELD OF THE INVENTION

This disclosure relates to systems, compounds and methods for stem cell delivery. More specifically, the disclosure relates to a system for promoting tissue regeneration, the system comprising a plurality of stem cells coated with at least one or a plurality of dendrimer nanocarriers that specifically bind to an adhesion molecule. Additionally, the disclosure relates to methods for delivering stem cells to damaged or diseased tissue for regeneration of the tissue.

### BACKGROUND

Mesenchymal Stem Cells (MSC) hold substantial promise as cancer therapeutics. Success of regenerative therapies relies heavily on the precise and efficient engraftment of stem cells into the target tissues. Currently, the most common stem cell therapy is direct injection into diseased tissue. However, this technique is complicated by several factors, including tissue physical pressure from forced inoculation and lack of sufficient nutrition and oxygen. There is a need in the art for improved methods for delivering stem cells to tissues and organs for therapeutic purposes.

### SUMMARY

This disclosure relates to systems and methods of stem cell delivery. In one embodiment, the disclosure provides a system for promoting tissue regeneration, the system comprising a plurality of stem cells coated with at least one or a plurality of dendrimer nanocarriers that specifically bind to an adhesion molecule.

In one aspect of the embodiment the plurality of stem cells are mesenchymal stem cells. In another aspect the one or plurality of dendrimer nanocarriers that specifically bind to an adhesion molecule comprise lymphocyte function-associated antigen-1 (LFA-1) comprise an amino terminal-inserted-domain (1-domain). In another aspect, the plurality of dendrimer nanocarriers comprise polyamido amine (PAMAM) dendrimer nanoparticles.

In yet another aspect, the adhesion molecule is an ICAM-1 molecule located on an activated endothelium cell at the inflamed periablation margins in liver, kidney heart, lung, intestinal, brain, or vasculature.

In another embodiment is a method to promote tissue regeneration, the method comprising: administering the system to a subject with liver disease for tissue regeneration following ablation therapy. The liver disease may be hepatitis A, hepatitis B, hepatitis C, hepatic cancer, hepatocellular carcinoma, fatty liver disease, cirrhosis, Alagille syndrome, Alcohol-related liver disease, Alpha-1 Antitrypsin Deficiency, Autoimmune hepatitis, liver tumors, billary artesia, Crigler-Najjar Syndrome, Galactosemia, Gilbert Syndrome, heomochromatosis, hepatic encephalopathy, Hepatorental syndrome, liver cysts, primary Sclerosing Cholangitis, Progressive Familial Intrahepatic Cholestasis. Reye Syndrome, Type I Glycogen Storage Disease, hemochromatosis, or Wilson disease

In yet another aspect the system is administered to a subject who has solid organ damage related to disease, trauma, or kidney disease including kidney cancer, chronic kidney disease, renal stenosis, nephropathy, glomerulonephritis, or kidney failure. In a further aspect, the system is administered to a subject having cardiac, vascular, or pulmonary conditions or related disease.

In a further embodiment is an in vitro method of promoting tissue regeneration, wherein a cell is contacted with the system for tissue regeneration.

### BRIEF DESCRIPTIONS OF THE DRAWINGS

FIG. 1 shows a schematic of a nanocarrier-directed systemic cell delivery system.

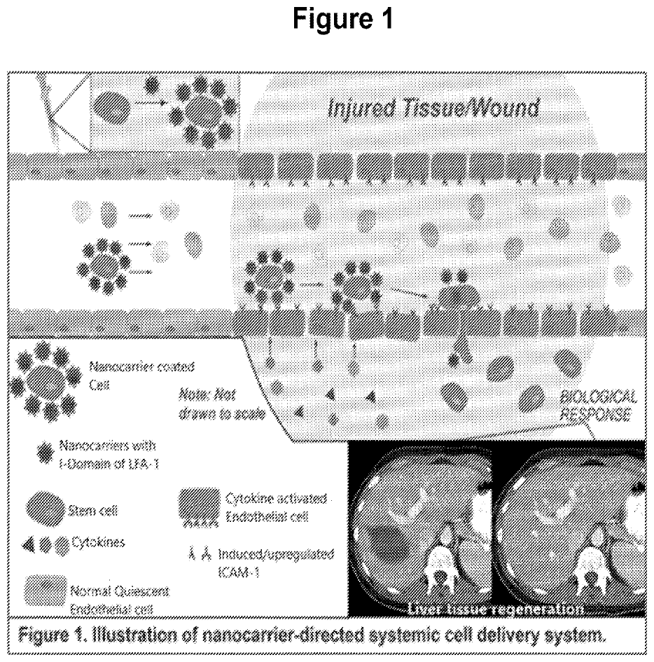

FIG. 2 shows an example of the rim of inflammation seen at the periablation margins as visualized by PET scan done 24 hours after pancreatic ablation.

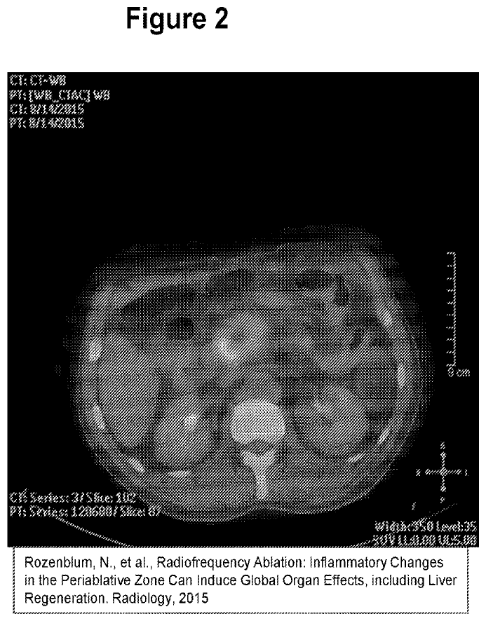

FIG. 3 shows CT liver ablation volume changes from Day 0 to Day 30 in test (left bar) and control (right bar) animal.

FIG. 4 shows contrast enhanced CT scan of the liver from a test animal. Scan immediately after ablation on day 0 (left) and 30 day scan (right), depicts the change in size of the ablation cavity which is outlined in yellow, red and green

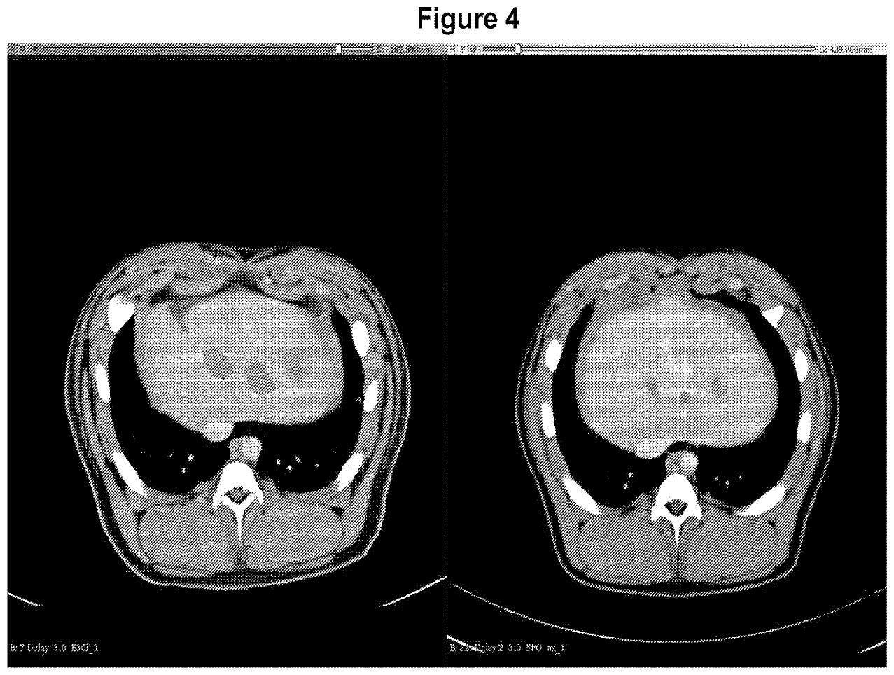

FIG. 5 shows the contrast enhanced CT scan of the liver from a test animal from day 0 (left) and day 30 (right) with the corresponding 3D volumetric reconstructions of the ablation cavities above the CT images.

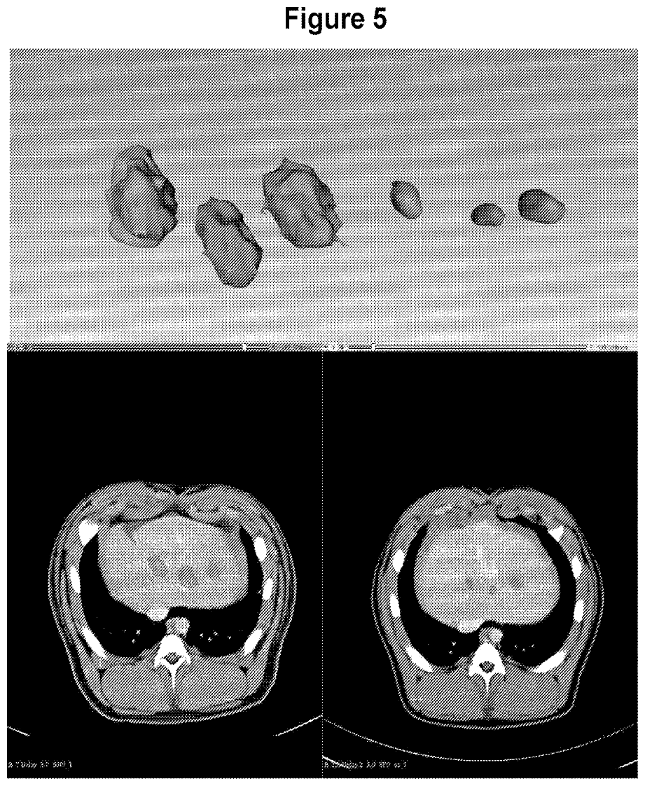

FIG. 6 shows cellular proliferation at margins of the ablation zone as identified by Ki67 count in test (left bar) and control (right bar) animals.

### DETAILED DESCRIPTION

This disclosure relates to systems, compounds and methods for stem cell delivery. More specifically, the disclosure relates to a system for promoting tissue regeneration, the system comprising a plurality of stem cells coated with at least one or a plurality of dendrimer nanocarriers that specifically bind to an adhesion molecule. Additionally, the disclosure relates to methods for delivering stem cells to damaged or diseased tissue to promote or aid in regeneration of the tissue.

As utilized in accordance with this disclosure, the following terms, unless otherwise indicated, shall be understood to have the following meanings. Unless otherwise required by context, singular terms shall include pluralities and plural terms shall include the singular.

Additionally, singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a meaningful range of a value. The allowable variation encompassed by the term “about” or “approximately” depends on the particular system under study, and can be readily appreciated by one of ordinary skill in the art.

### Hepatocellular Cancer (HCC)

Hepatocellular cancer (HCC) is the fifth most diagnosed cancer and second leading cause of cancer related death in the world (Jemal, A., et al., Cancer J Clin, 2011, 61(2): 69-90). Image guided radiofrequency ablation (RFA) is a minimally invasive curative treatment option for HCC. RFA provides a targeted heated treatment that ablates tumor cells and is effective in patients that meet criteria for RFA. Multiple randomized controlled trials have shown its efficacy to be equivalent to that of resection for HCC (Fang, Y., et al., J Gastroenterol Hepatol, 2014, 29(1): 193-200; Ng, K. K. C., et al., Br J Surg, 2017, 104(13): 1775-1784; Feng, K., et al., J Hepatol, 2012, 57(4): 794-802; Chen, M. S., et al., Ann Surg, 2006, 243(3): 321-328. However, severe liver dysfunction associated with cirrhosis limits the use of thermal ablation in many patients due to the potential for complete liver failure from tissue loss following ablation (Ryder, S. D., Gut. 2003, 52 Suppl 3: 1-8; Koda, M., e al., Hepatol Res, 2004. 29(1): 18-23; Kuroda, H., et al., Hepatol Res, 2010, 40(5): 550-554). Liver tissue regeneration using stem cells following ablation would help to restore liver functions and additionally allow more patients to meet minimum requirements for ablation.

In 2012, the global burden of HCC exceeded 14 million cases, with HCC predicted to affect over 22 million people over the next two decades (Stewart B W, World Cancer Report 2014). Among all cancers, HCC is one of the fastest growing causes of death in the US and poses a significant economic burden on healthcare (Ghouri, Y. A., et al., J Carcinog, 2017, 16: 1). The incidence rate of HCC has increased from 1.4 cases per 100,000 people between 1976-1980 to 6.2 cases per 100,000 people in 2011 (El-Serag, H. B. and A. C. Mason, N Engl J Med., 1999, 340(10): 745-750). Cirrhosis with associated liver dysfunction is an underlying diagnosis in 80-90% of these cases (Fattovich, G., et al., Gastroenterology, 2004, 127 (5 Suppl 1): S35-S50).

Chronic liver diseases resulting in cirrhosis predisposes patients to HCC. Regardless of the etiology, the hepatic response to injury involves formation of fibrous septae with cirrhosis representing the late stage of progressive hepatic fibrosis. Cirrhosis with varying degrees of liver dysfunction is an underlying diagnosis in 80-90% of cases of HCC worldwide (Id).

### Current Treatments for HCC

Surgical resection is an effective treatment for the management of HCC. However, surgery is only suitable for 9-27% of patients with HCC due to factors such as poor hepatic reserve, multifocal distribution of tumors, or other co-morbidities (Fong. Y., et al., Ann Surg, 1999, 229(6): 790-800; Lai, E. C., ci al., Ann Surg, 1995, 221(3): 291-298). Additionally, liver resection is associated with significant morbidity.

Minimally invasive image guided percutaneous thermal ablation is an alternative option in the management of liver tumors which obviates the need for surgical resection. Randomized control trials have shown similar efficacy of percutaneous thermal ablation for the treatment of HCC compared to surgery (Id.).

Percutaneous ablation is performed by placing specialized probes into a tumor under image guidance (e.g. computerized tomography, ultrasound, or magnetic resonance imagining), to deliver heat, electricity or chemicals directly into the tissues causing cell death. The most commonly used thermal ablative technique is radiofrequency ablation (RFA).

RFA probes deliver high frequency alternating current to tissues. In response, the tissue ions attempt to follow the change in the direction of the alternating current at radiofrequency resulting in frictional heating of surrounding tissue. Temperatures at approximately 60° C. result in cellular death and coagulation necrosis of area surrounding the radiofrequency probe. Effective tumor treatment recommendations indicate the ablation zone should encompass the entire tumor and a circumferential margin of approximately 5 to 10 mm (Sainani, N. I., et al., Am J Roentgenol, 2013, 200(1): 184-93.

Patients with large tumors or severely compromised liver functions (Child Pugh stage C) are not candidates for radio frequency ablation as liver dysfunction limits the use of thermal ablation due to potential post procedure complete liver failure from tissue loss (Ryder, S. D., Gut, 2003, 52 Suppl 3: 1-8: Koda, M., et al., Hepatol Rev. 2004, 29(1): 18-23; Kuroda, H., et al., Hepatol Res, 2010, 40(5): 550-544. The incidence of liver failure following ablation is estimated to be 0.2% to 4.3% (Fonseca, A. Z., et al., World J Heptol, 2014, 6(3): 107-113) and Child Pugh scores increase significantly following ablation at 6 and 12 months (Kuroda, H., et al., Hepatol Res, 2010 40(5): 550-554). These factors underscore the clinical necessity for liver tissue regeneration modalities following ablation that can prevent liver failure and subsequent mortality.

MSC are ideally suited for liver tissue regeneration. In vitro, MSC have been shown to differentiate into hepatocyte-like cells with functional properties such as albumin and urea production, glycogen storage, LDL uptake, and phenobarbital-induced cytochrome p450 expression (Talens-Visconti, R., et al., World J Gastroenterol, 2006, 12(36): 5834-5845; Schwartz, R. E., et al., J Clin Invest. 2002, 109(10): 1291-1302). Additionally, MSC secrete several anti-fibrotic molecules such as hepatocyte growth factor while in vivo, hepatic differentiation of MSC has been demonstrated in both animals and humans (Berardis, S., et al., PLoS One, 2014, 9(1): e86137; Alison, M. R., et al., Nature, 2000, 406(6793): 257: Chamberlain, J., et al., Hepatology, 2007, 46(6): 1935-1945).

Bone marrow derived MSC have been used as a therapeutic option to promote tissue regeneration in various organs (Brown, C., et al., J Tissue Eng Regen Med, 2019, 13(9): 1738-1755; Kang, S. H., et al., Gut Liver, 2019). However, success of regenerative therapies is intricately linked to the precise and efficient homing and engraftment of stem cells into target tissues with direct injection of cells into diseased or damaged tissue being the most common route of stem cell administration. Despite this, direct injection may not be an optimum route as the cells may not survive due to factors such as tissue physical pressure from forced inoculation or lack of sufficient nutrition and oxygen as forcibly inoculated cells may not stay close to vessels.

Animal studies show mixed regenerative results with the use of hematopoietic or MSC for the regeneration of liver tissue however the results have been mixed. For instance, bone marrow derived mesenchymal stem cells regenerate liver tissue in a 70% hepatectomy rodent model (Liu, Y., et al., Stem Cells Int, 2018, 2018: 7,652,35; Li, D. L., et al., Pathobiology, 2013, 80(5): 228-234).

However, in the same model, MSC infusion did not significantly affect liver function, proliferative index, or number of mitoses (Alves, A. K. S., et al., Acta cir Bras, 2017, 32(7): 515-522). In addition, early human trials have also explored the use of hematopoietic or MSC in liver disease. Stems cells have been shown to improve liver tissue regeneration following liver resection and transarterial chemoembolization (Ismail, A., et al., J Gastrointest Cancer, 2011, 42(1): 11-19; Ismail, A., et al., J Gastrointest Cancer, 2010, 41(1): 17-23). Further, autologous CD133+ bone marrow stem cell treatment resulted in increased liver volume allowing for earlier surgery to remove liver metastases (Furst. G., et al., Radiology, 2007, 243(1): 171-179).

### Systems and Methods for Tissue Regeneration

In one embodiment, disclosed herein is a system for promoting tissue regeneration, the system comprising a plurality of stem cells coated with at least one or a plurality of dendrimer nanocarriers that specifically bind to an adhesion molecule.

In particular embodiments, the plurality of stem cells are embryonic stem cells, tissue specific somatic cells, or induced stem cells. Ina preferred embodiment the plurality of stem cells are mesenchymal stem cells.

A “nanocarrier” is a nanomaterial, or materials, with a single unit that ranges in size from 1 to 1000 nanometers, used to carry or transport another substance such as a drug or therapeutic modality. Examples of nanocarriers include polymer conjugates, polymeric nanoparticles, lipid-based carriers, dendrimers, carbon nanotubes, and gold nanoparticles (nanoshells and nanocages), liposomes and micelles. The material composition of the nanocarrier provides properties required to transport a wide range of hydrophobic and hydrophilic drugs, including both. One of skill in the art will understand that the another substance or drug modality to be transported encompasses a wide range of compounds, proteins, or therapeutic modalities. Nanocarriers deliver drugs via passive targeting, active targeting, pH specificity, and/or temperature specificity.

A “nanomaterial” is defined by the International Organization for Standardization as “material with any external dimension in the nanoscale or having internal structure or surface structure in the nanoscale”, with “nanoscale” defined as the “length range approximately from 1 nm to 100 nm”. This includes both nano-objects, which are discrete pieces of material, and nanostructured materials, which have internal or surface structure on the nanoscale; a nanomaterial may be a member of both these categories. (ISO/TS 80004-3:2010, Nanotechnologies—Vocabulary—Part 3: Carbon nano-objects).

A nanomaterial can also be a natural, incidental or manufactured material containing particle, in an unbound state or as an aggregate or as an agglomerate and for 50% or more of the particles in the number size distribution, one or more external dimensions is in the size range 1 nm-10 nm. In specific cases and where warranted by concerns for the environment, health, safety or competitiveness the number size distribution threshold of 50% may be replaced by a threshold between 1% to 50%″ (European Commission definition, adopted) 18 Oct. 2011).

In particular embodiments, the nanocarrier comprises polyamido amine (PAMAM) dendrimer nanoparticles. Dendrimer Nanocarriers are polymeric chemical structures that can be used to contain, transport and deliver drugs or compounds of interest. The architecture of the PAMAM nanocarrier comprises a channel or structure in which compounds, drugs, or cell therapies that can be encapsulated within the PAMAM architecture. Further, one of skill in the art will understand that the PAMAM can be associated on the dendrimer surface. Dendrimer nanocarriers are characterized by high solubility, stability and encapsulation of a wide range of compounds. Furthermore, as disclosed herein, the dendrimer can be modified to allow targeting to tissues of interest

In particular embodiments, the dendrimer nanocarrier comprises lymphocyte function-associated antigen-1 (LFA-1). In particular embodiments, the lymphocyte function-associated antigen-1 (LFA-1) comprises the amino terminal-inserted-domain (1-domain).

In particular embodiments, the dendrimer nanocarrier specifically binds to an adhesion molecule. Cell adhesion molecules are glycoproteins found on the cell surface and extracellular matrices and paly roles in homophilic and heterophilic protein-protein interactions during cell adhesion. In some embodiments the cell adhesion molecule is intercellular cell adhesion molecule-1 (ICAM-1). ICAM-1 is upregulated in endothelial cells in response to injury (Frank, P. G. and M. P. Lisanti, Am J Physiol Heart Circ Physiol, 2008, 295(3): 14926-1927). Pro-inflammatory cytokines can also induce vascular expression of ICAM-1 (Wyble, C. W., et al., J Surg Res, 1997, 73(2): 107-112: Liu, Z. J., et al., Ann Surg, 2010, 252(4): 625-634; Lasky, L. A, Science, 1992, 258(5084): 964-969. ICAM-1 interacts with its counterpart adhesion molecule, lymphocyte function-associated antigen-1 (LFA-1; Wee, H., I., Exp Mol Med, 2009 41(5): 341-348; Witkowska, A. M. and M. H. Borawska, Eur Cytokine Netw, 2004, 15(2): 91-98). ICAM-1/LFA-1 are known to be involved in the interaction between leukocytes and the endothelial cells for leukocyte trans-endothelial migration in inflammation (Wee, H., et al., Exp Mol Med. 2009, 41(5): 341-348). ICAM-1 is a cell surface adhesion molecule and only minimally expressed in the quiescent endothelial cells. Levels of ICAM-1 on the endothelium can be increased in response to pathological stimuli. The α-subunit of LFA-1 consists of an amino terminal-inserted-domain (I-domain), which is essential for LFA-1 binding to ICAM-1 (Landis, R. C. et al., J Cell Biol, 1994, 126(2): 529-537; Edwards. C. P., et al., J Biol Chem. 1995, 270(21): 12635-12640; Huang, C. and T. A. J Biol Chem, 1995, 270(32): 19008-19016; Manikwar, P., et al., Theranostics, 2011, 1: 277-289). I-domain has two important sites for modulation of binding to ICAM-1 and is responsible for the interaction between LFA-1 and ICAM-1 (Id.). In some embodiments, the cell adhesion molecule can be one or more of ICAM-1, ICAM-2, ICAM-3, ICAM-4, ICAM-5, and their fragments.

In particular embodiments, the ICAM-1 molecule is located on an activated endothelium cell. In particular embodiments, the activated endothelium cell at the inflamed periablation margins is located in the liver, kidney heart, lung, intestinal, brain, or vasculature. In one embodiment, the endothelium cell is located in diseased tissue including kidney tissue following thermal ablation or the liver following transarterial chemo and radio embolizations.

One of the key changes with inflammation is the change in permeability of the endothelial cell lumen. Normally, luminal endothelial cells form a natural barrier between the blood and surrounding tissue and under physiological conditions, is a tight impermeable barrier. In response to injury, a variety of cytokines/chemokines, for example, SDF-1α, TNF-α, and IL-1, are released into tissue, and the local endothelium is stimulated. The cytokine release results in upregulation, and/or activation of a unique panel of cell adhesion molecules (CAMs), including ICAM-1, VCAM-1, selectin and integrin in the endothelium of the local tissue. As a result, the activated endothelium becomes highly permeable.

In particular embodiments, disclosed herein are methods for promoting tissue regeneration in a subject. The term “subject” is intended to include humans and non-human animals, particularly mammals.

In some embodiments, the subject has a liver disease, including but not limited to hepatitis A, hepatitis B, hepatitis C, hepatic cancer, hepatocellular carcinoma, fatty liver disease, cirrhosis, Alagille syndrome. Alcohol-related liver disease, Alpha-1 Antitrypsin Deficiency, Autoimmune hepatitis, liver tumors, billary artesia, Crigler-Najjar Syndrome, Galactosemia, Gilbert Syndrome, heomochromatosis, hepatic encephalopathy. Hepatorental syndrome, liver cysts, primary Sclerosing Cholangitis, Progressive Familial intrahepatic Cholestasis, Reye Syndrome, Type 1 Glycogen Storage Disease, hemochromatosis, and/or Wilson disease. Further, a system for promoting tissue regeneration, as disclosed herein, is beneficial in individuals suffering from reduced liver capacity related to trauma, disease, or injury to other bodily organs.

The system disclosed herein could be modified for use in a wide range of conditions or diseases affecting additional organs. In one aspect the disease is kidney disease including, but not limited to, kidney cancer, chronic kidney disease, renal stenosis, nephropathy, glomerulonephritis, kidney failure, renal angiomyolipoma, adenoma, fibroma, lipoma, or oncocytoma. In another aspect the system can be adapted for use in treatment of cardiac, vascular, or pulmonary conditions or diseases, including but not limited to cardiac insufficiency, heart disease, pulmonary fibrosis, and vascular damage.

In another aspect, the system could be used as a treatment modality for solid organ non-liver cancers or disease characterized by tissue damage. This includes cancers of the bone, lung, heart, kidney, pancreas, lung, small intestine, or colon.

The terms “administration” or “administering” as used herein refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

The terms “pharmaceutical composition” or “therapeutic composition” as used herein refer to a compound or composition capable of inducing a desired therapeutic effect when properly administered to a subject. In some embodiments, the disclosure provides a pharmaceutical composition comprising a pharmaceutically acceptable carrier and a therapeutically effective amount of at least system of the disclosure.

The terms “pharmaceutically acceptable carrier” or “physiologically acceptable carrier” as used herein refer to one or more formulation materials suitable for accomplishing or enhancing the delivery of one or more system of the disclosure.

When used for in vivo administration, the formulations of the disclosure should be sterile. The formulations of the disclosure may be sterilized by various sterilization methods, including, for example, sterile filtration or radiation. In one embodiment, the formulation is filter sterilized with a pre-sterilized 0.22-micron filter. Sterile compositions for injection can be formulated according to conventional pharmaceutical practice as described in “Remington: The Science & Practice of Pharmacy,” 21st ed., Lippincott Williams & Wilkins, (2005). The formulations can be presented in unit dosage form and can be prepared by any method known in the art of pharmacy. Actual dosage levels of the active ingredients in the formulation of this disclosure may be varied so as to obtain an amount of the active ingredient, which is effective to achieve the desired therapeutic response for a particular subject, composition, and mode of administration, without being toxic to the subject (e.g., “a therapeutically effective amount”). Dosages can also be administered via continuous infusion (such as through a pump). The administered dose may also depend on the route of administration. For example, subcutaneous administration may require a higher dosage than intravenous administration.

The methods and systems disclosed herein, advantageously direct MSC to target tissue by way of recognition of, and association with, adhesion molecules expressed on the activated endothelium of the injured tissues. Once anchored on the activated endothelium, nanocarrier-coated cells extravasate and home to the targeted tissues to execute their effects on tissue repair and regeneration. This eliminates the need for direct injection of MSC into diseased and/or damaged tissue.

In yet another embodiment is an in vitro method of promoting tissue regeneration is provided wherein a cell is contacted with the system for promoting tissue regeneration. “Contacting” as used herein means to bring a cell in proximity of the system and can include touching of the cell and system or wherein the cell and system are in close approximation of each other but not touching. Close approximation can include a distance that allows for communication between the cell and system, typically a distance of micrometers or nanometers.

### EXAMPLES

The Examples that follow are illustrative of specific embodiments of the disclosure, and various uses thereof. They are set forth for explanatory purposes only and should not be construed as limiting the scope of the disclosure in any way.

### Example 1. Animals

Twelve Yorkshire pigs (male n=6; female n=6) were divided into test and control groups, each group consisting of 3 male pigs and 3 female pigs. Power calculation was based on the assumption that the number of ablations within a pig is four and success rates of liver tissue regeneration in control and treatment group are 5% and 85%, respectively. A correlation assumption was made that among ablation sites the intra-pig correlation is 0.5, thereby equating that 12 pigs will give 80% power to detect a difference of 80%.

### Example 2. Bone Marrow Cell Culture

Bone marrow (BM) aspirate was obtained from the long hind bone of two animals using a standard aspiration kit. Aspirates were collected in heparin-coated glass tubes and washed twice with phosphate buffered saline (PBS). A total of 10⁷ BM cells were cultured in 10 cm petri-dish with Dulbecco's Modified Eagle Medium/Nutrient Mixture F-12 (DMEM/F-12) from Gibco (cat. 11320082) containing 20% Fetal Bovine Serum (FBS, HyClone, cat. SH13091003) and Penicilin/Streptomycin Antibiotic from Gibco (cat. 15140-122). Non-adherent cells were moved by periodical changes of medium, every 3 days. When cells reached approximately 70% confluency, MSC were transduced with eGFP/Lentivirus. Two days after transduction, eGFP+/MSC were detached from culture dishes by Trypsin-EDTA (Corning) and washed twice by PBS and re-suspended in complete DMEM/F-12.

### Example 3. Nanocarriers

G5-dendrimers are purchased from Dendritech (Midland, Mich.) and recombinant I-Domain of LFA-1 fragments were produced in E. coli and purified. The ratio between the acetic anhydride and the dendrimer was to achieve acetylation of ˜30% of total amino groups. Reactions were carried out at room temperature for 24 hours under argon gas. Reaction mixtures were dialyzed in PBS and then water. The conditions for the preparation of the dendrimer/protein complex that remains “parked” on the cell surface for ˜4 h were optimized. Ac-G5 dendrimers were complexed with I-Domain of LFA-1 fragments. Mixtures were incubated for 15 min at room temperature to allow for formation of nanocarriers. The nanocarriers were washed and re-suspended in DMEM. Nanocarriers (1 mL) were mixed with 1×10⁶ MSC and incubated for 20 min at room temperature and mixed every five minutes. Nanocarrier-coated MSC were centrifuged 1200 rpm for 5 minutes and re-suspended in sterile PBS in preparation for injection.

### Example 4. Radio Frequency Ablation and Stem Cell Delivery

Radio frequency ablation (RFA) was performed with a Covidien Cool-Tip ablation system (Covidien, Boulder, Colo.). Procedures were performed under general anesthesia followed by a non-contrast computerized tomography abdominal scan. RFA probes were placed with ultrasound guidance. Based on liver size, 2 to 4 ablations (2 cm target dimeter) were performed in each animal. Target ablation size was set using recommend manufacturer parameters and kept uniform for all animals. Immediately following ablation, a contrast enhanced triple phase CT scan of the liver was obtained.

Stem cell delivery (10⁶ cells/porcine) was performed the day after the RFA, a timeframe when periablational inflammation is expected to peak. Stem cell delivery was achieved via right groin access. Reverse curve catheter was used to select the celiac trunk and the hepatic artery was selected using a microcatheter. After confirming catheter position, approximately 10⁶ MSC coated with nanocarriers suspended in 5 mls of PBS were injected into the hepatic artery.

Ten animals completed the entire 30 day study protocol. Blood tests, including liver function panel, renal function panel and complete blood count were performed on all animals on day 0 (Prior to ablation), day 15, and day 30. All animals also received a contrast enhanced triple phase liver CT scan on day 30 under general anesthesia after which animals were euthanized and livers explanted

### Example 5. CT Scan Analysis

CT scans were analyzed to compare the ablation cavity volume changes from day 0 to day 30 in test and control animals. Ablation cavity was defined as the ablated area that did not enhance on contrast CT scan, denoting a lack of perfusion. Image analysis was performed using 3D Slicer (Brigham Women's Hospital, Boston, Mass.), a free, open source medical image computing software (Fedorov, A., et al., Magn Reson Imaging (2012) 30(9): 1323-41). Boundaries of non-enhancing ablation zones were manually segmented by an experienced interventional radiologist. Ablation zones were then reconstructed in three dimensions and volumes computed using 3D Slicer.

Cell proliferation at the margins of the ablation zone was measured using Ki67 staining, immunostaining was used to identify GFP positive cells to determine the amount and location of the recruited GFP positive MSC. Liver function at days 15 and 30 was compared between test and control groups. Statistical analysis was performed using SPSS software (IBM). Paired sample T-tests were used for analysis of continuous variables with significance set at p=0.05.

### Results

### Tissue Regeneration

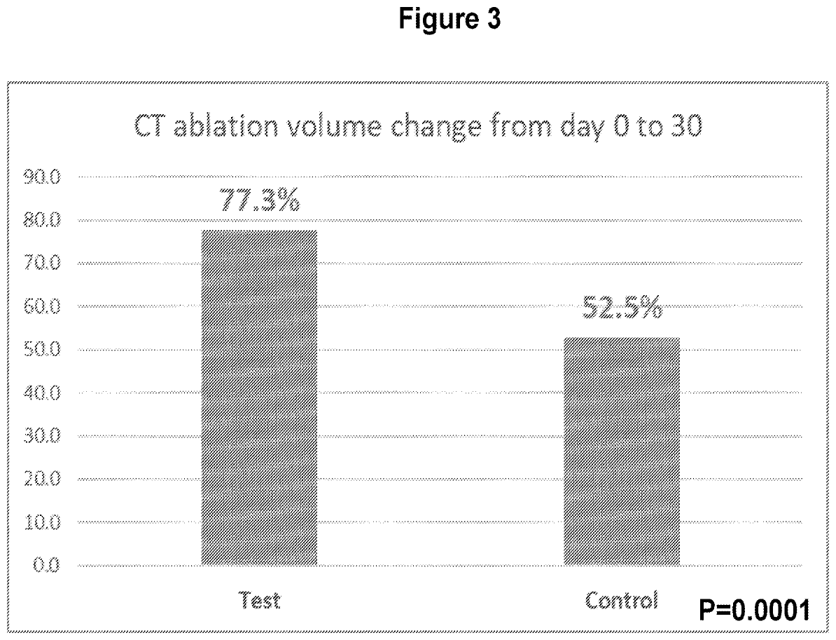

Reduction in size of the ablation cavity on contrast enhanced CT scans was the primary outcome used to assess liver tissue regeneration. The mean ablation volume at 0 and 30 days for the control group was 5.6 cm³ (SD 2.4) and 2.4 cm³ (SD 0.9) respectively. The mean ablation volume at 0 and 30 days for the test group was 6.4 cm³ (SD 3.1) and 1.2 cm³ (SD 0.8) respectively. Ablation cavity in the test group decreased by 77.3% (SD 12.1) from day 0 to 30 compared to 52.5% (SD 22.5) in the control group (FIG. 3; p=0002).

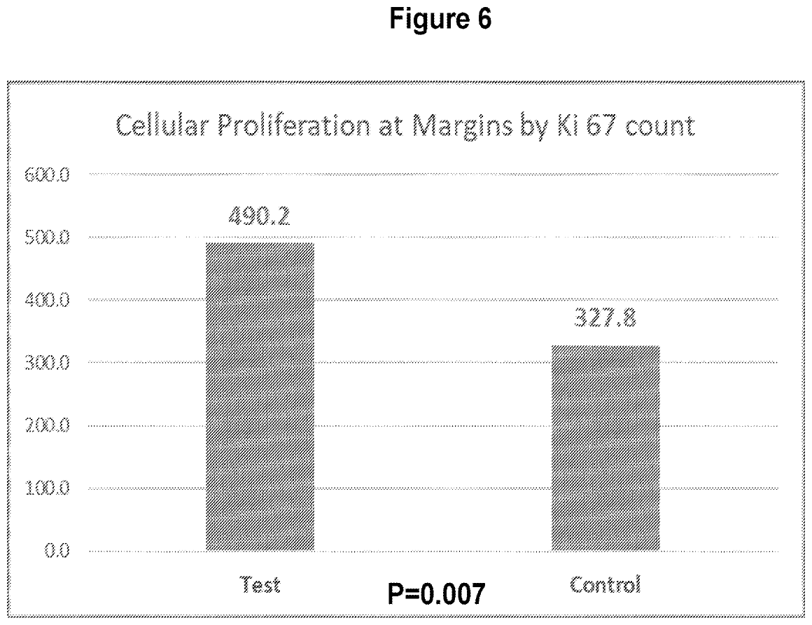

Tissue regeneration at the margins of the ablation cavity was assessed using the cellular proliferation marker Ki67. Mean Ki67 count for the test group was 49 (0.2 (SD 170.1) per 20× field compared to 327.8 (SD 133.3) per 20× field for controls (FIG. 6; p=0.007). GFP staining was used to identify the lineage of cells, i.e. those that originated from the infused MSCs. GFP positive cells were identified throughout the regenerated tissues at the ablation margins. The mean depth of the penetration from the margin of the ablation for the GFP positive cells was 300.6μ(SD 48.6).

In the control group, aspartate amino transferase (AST) increased by 25.5% (SD 0.29) from day 0 to 15 whereas AST was not increased in the test group (mean change −10.2%; SD 0.11; p=0.02. Complete blood count, renal function test results, and measurements of liver functions were not different between groups throughout the duration of the experiment.

The results demonstrated that nanocarrier mediated stem cell delivery significantly improved liver tissue regeneration at the margins following thermal ablation. This strategy makes curative treatment of ablation available to a large number of patients with hepatocellular cancer and severe liver dysfunction, who otherwise would have no therapeutic option. The nanocarrier mediated stem cell delivery is a promising and versatile tool with wide application in regenerative medicine. The platform technology and delivery system can be used for targeted delivery of any type of cells to specific tissues. For this purpose, appropriate adhesion molecules expressed in a specific tissue or organ can be selected and a nanocarrier created using the counterpart adhesion molecule, which can then be attached to the cell to be delivered

Thermal ablation of renal cancers is widely used as a therapeutic option for stage 1 renal cell carcinoma. The same targeted stem cell delivery strategy using ICAM-1/LFA-1 can be applied for regeneration of kidney tissues in such cases. Nanocarrier mediated stem cell delivery with same or novel adhesion molecule pairs can be used in these cases for regeneration of liver tissue. Similarly, transarterial chemoembolization and radioembolization are widely used for liver cancer treatment where this same strategy of nanocarrier mediated targeted stem cell delivery can be utilized for tissue regeneration.

As described in the Examples and shown in the accompany drawings, methods for using nanocarrier mediated stem cell delivery via adhesion molecule to specifically direct stem cells to sites of injury significantly improved liver tissue regeneration at the margins following thermal ablation. One of skill in the art will understand the additional clinical applications for this technology including inter alia use in targeted delivery of stem cells for regeneration of kidney following thermal ablation, and in the liver following transarterial chemo and radio embolizations.

What is claimed is:

1: A method to promote tissue regeneration, the method comprising: administering a system comprising a plurality of stem cells coated with a plurality of dendrimer nanocarriers that specifically bind to an adhesion molecule to a subject, wherein the subject has liver disease.

2: The method of claim 1, wherein the liver disease is hepatitis A, hepatitis B, hepatitis C, hepatic cancer, hepatocellular carcinoma, fatty liver disease, cirrhosis, Alagille syndrome, Alcohol-related liver disease, Alpha-1 Antitrypsin Deficiency, Autoimmune hepatitis, liver tumors, billary artesia, Crigler-Najjar Syndrome, Galactosemia, Gilbert Syndrome, heomochromatosis, hepatic encephalopathy, Hepatorental syndrome, liver cysts, primary Sclerosing Cholangitis, Progressive Familial Intrahepatic Cholestasis, Reye Syndrome, Type I Glycogen Storage Disease, hemochromatosis, or Wilson disease.

3: A method to promote tissue regeneration, the method comprising: administering a system comprising a plurality of stem cells coated with a plurality of dendrimer nanocarriers that specifically bind to an adhesion molecule to a subject, wherein the subject has solid organ damage related to disease, trauma, or injury.

4: A method to promote tissue regeneration, the method comprising: administering a system comprising a plurality of stem cells coated with a plurality of dendrimer nanocarriers that specifically bind to an adhesion molecule to a subject, wherein the subject has kidney disease.

5: The method of claim 4, wherein the kidney disease is kidney cancer, chronic kidney disease, renal stenosis, nephropathy, glomerulonephritis, kidney failure, renal angiomyolipoma, adenoma, fibroma, lipoma, or oncocytoma.

6: A method to promote tissue regeneration, the method comprising: administering a system comprising a plurality of stem cells coated with a plurality of dendrimer nanocarriers that specifically bind to an adhesion molecule to a subject, wherein the subject has cardiac, vascular, or pulmonary conditions or related disease.

7: An in vitro method of tissue regeneration, the method comprising: contacting a cell with a system comprising a plurality of stem cells coated with a plurality of dendrimer nanocarriers that specifically bind to an adhesion molecule.

8: The method of any one of claims 1-7, wherein the plurality of stem cells are mesenchymal stem cells.

9: The method of any one of claims 1-7, wherein the one or plurality of dendrimer nanocarriers that specifically bind to an adhesion molecule comprise lymphocyte function-associated antigen-1 (LFA-1).

10: The method of any one of claims 1-7, wherein the plurality of dendrimer nanocarriers comprise polyamido amine (PAMAM) dendrimer nanoperticles.

11: The method of any one of claims 1-7, wherein the adhesion molecule is ICAM-1.

12: The method of claim 11, wherein the ICAM-1 molecule is located on an activated endothelium cell.
